(19) United States
(12) Patent Application Publication
(10) Pub. No.: US 2026/0013938 A1
(43) Pub. Date: Jan. 15, 2026

(54) TISSUE PENETRATING DEVICE TIPS

(71) Applicant: INTUITIVE SURGICAL OPERATIONS, INC., Sunnyvale, CA (US)

(72) Inventors: Stephen J. Blumenkranz, Los Altos, CA (US); Hans Valencia, San Jose, CA (US)

(21) Appl. No.: 19/337,449

(22) Filed: Sep. 23, 2025

Related U.S. Application Data
(63) Continuation of application No. 16/670,846, filed on Oct. 31, 2019.
(60) Provisional application No. 62/754,976, filed on Nov. 2, 2018.

(51) Int. Cl. A61B 18/18; A61B 17/32; A61B 17/34; A61M 25/00

(52) U.S. Cl. CPC A61B 18/1815; A61B 2017/320056; A61B 2017/3454; A61B 2018/183; A61B 2018/1861; A61B 2018/1869; A61M 25/0068

(57) ABSTRACT

An ablation instrument comprises a cable, a conductive antenna body coupled to the cable and configured to deliver ablative energy to tissue, and a tip having a cross-sectional diameter of less than 5 mm. The tip comprises a blade configured to cut a slit in the tissue. The blade comprises a plurality of cutting edges, and each cutting edge of the plurality of cutting edges has a width between 30% and 50% of the cross-sectional diameter of the tip.

### CROSS-REFERENCE TO RELATED APPLICATIONS

This application claims priority to U.S. Provisional Application No. 62/754,976, filed on Nov. 2, 2018, which is incorporated herein by reference in its entirety.

### FIELD

Embodiments described herein generally relate to tissue penetrating device tips. Specifically, embodiments described herein relate to tissue penetrating device tips that reduce penetration force.

### BACKGROUND

Treatment of various conditions may require diagnosis and/or treatment including delivery of drugs, delivery of implants, delivery of ablative energy or removal of tissue. While benign tissue may be removed, it is often necessary to detect and remove or destroy a cancerous tumor. In particular, destroying a tumor during early stages of disease may ensure the tumor does not grow large enough to interfere with the body's functions and also reduces the likelihood of the cancer spreading throughout the body, which can be life-saving.

Medical devices may be delivered to the location of tissue to be treated (e.g., through a catheter) to diagnose, treat, and/or alter the tissue. In the case of ablation, the medical device may penetrate the tissue and emit energy from an antenna or probe located at or near the center of the tissue to be treated.

While it is desirable to destroy tumors when they are still small (e.g., largest dimension of less than 3 cm), penetrating smaller tumors presents challenges because they may be easily displaced. Thus, it can be difficult to ensure appropriate placement of the ablation antenna to ablate the tumor or other tissue.

### BRIEF SUMMARY

Some embodiments described herein relate to medial instrument tips for penetrating tissue. In some embodiments, a medical instrument tip may include a body having a proximal portion with a diameter of less than 5 mm, and a blade distal to the proximal portion of the body. The blade of the medical instrument tip may include a plurality of faces and a plurality of cutting edges, wherein each cutting edge of the plurality of cutting edges is formed by adjacent faces of the plurality of faces, and at least one cutting edge of the plurality of cutting edges may have a dihedral angle of less than 50 degrees. In some embodiments, the device tip may have a cross-sectional diameter of less than 5 mm. In some embodiments, the dihedral angle may be between 25 and 35 degrees. In some embodiments, the diameter of the proximal portion of the body may be less than 3 mm.

In some embodiments, at least one cutting edge may have a thickness of less than 1 micron. In some embodiments, the plurality of faces may include a plurality of concave faces. In some embodiments, the plurality of faces may include three faces or four faces. In some embodiments, each cutting edge of the plurality of cutting edges may have a dihedral angle of between approximately 15 degrees to 40 degrees.

In some embodiments, the body may include a cone shaped body, wherein the blade may include a flat blade tip, and the flat blade tip may be at least partially disposed in the cone shaped body. In some embodiments, the flat blade tip may extend distally out of the cone shaped body by less than 1 mm. In some embodiments, the medical instrument tip may further include a lubricant on one or more of the cone shaped body or the flat blade tip. In some embodiments, the flat blade tip may be secured in the cone shaped body by overmolding the cone shaped body around a portion of the flat blade tip.

Some embodiments described herein relate to ablation instruments. In some embodiments, an ablation instrument may include a cable, a conductive antenna body coupled to the cable and configured to deliver ablative energy to tissue, and a tip having a cross-sectional diameter of less than 5 mm. The tip of the ablation instrument may include a blade configured to cut a slit in the tissue, and the blade may include a plurality of cutting edges, and each cutting edge of the plurality of cutting edges may have a width between 30% and 50% of the cross-sectional diameter of the tip. In some embodiments, the tip may include a cone shaped body made of a high temperature plastic, wherein the blade may be partially disposed in the cone shaped body, and the blade may be made of a metal. In some embodiments, the tip may include grooves, and the conductive antenna body may include protrusions, wherein the protrusions of the conductive antenna body are configured to engage the grooves of the tip, and the tip and the conductive antenna body may be joined with a sealant to create a fluid tight seal.

Some embodiments described herein relate to systems for penetrating target tissue. In some embodiments, the system may include a catheter extendable to target tissue, and the catheter may include a working lumen having an inner diameter of less than 5 mm, and a device tip configured to pass through the working lumen of the catheter and penetrate the target tissue, wherein the device tip may include a cutting edge having a thickness of less than 1 micron and a dihedral angle of less than 50 degrees.

In some embodiments, the device tip may include a hollow ground tip. In some embodiments, the device tip may include a flat blade tip. In some embodiments, the device tip may include a plurality of cutting edges and a dihedral angle of each cutting edge of the plurality of cutting edges may be between 25 and 35 degrees. In some embodiments, the device tip may include a body portion having a cross-sectional diameter, and the device tip may include a plurality of cutting edges, wherein each cutting edge of the plurality of cutting edges may have a width of between 30% and 50% of the cross-sectional diameter.

### BRIEF DESCRIPTION OF THE DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows a cross-section view of an elongate flexible device with a tissue penetrating device tip within the elongate flexible device.

FIG. 2 shows a top view schematic of a tissue penetrating device tip according to some embodiments.

FIG. 3 shows a top view schematic of a tissue penetrating device according to some embodiments.

FIG. 4 shows a top view schematic of a tissue penetrating device according to some embodiments.

FIG. 5 shows a perspective view of a tissue penetrating device tip according to some embodiments.

FIG. 6 shows a perspective view of a tissue penetrating device tip according to some embodiments.

FIG. 7 shows a top view of a tissue penetrating device tip according to some embodiments.

FIG. 8 shows a cross-sectional view taken along line VIII-VIII in FIG. 7 of a tissue penetrating device tip according to some embodiments.

FIG. 9 shows a perspective view of a tissue penetrating device tip according to some embodiments.

FIG. 10 shows a top view of a tissue penetrating device tip according to some embodiments.

FIG. 11 shows a perspective view of a tissue penetrating device tip according to some embodiments.

FIG. 12 shows a perspective view of a cone for tissue penetrating device tip according to some embodiments.

FIG. 13 shows a perspective view of a flat blade tissue penetrating device tip according to some embodiments.

FIG. 14 shows a transparent perspective view of a cone shaped body for a flat blade tissue penetrating device tip of FIG. 13 according to some embodiments.

FIG. 15 shows a perspective view of a blade for a flat blade tissue penetrating device tip of FIG. 13 according to some embodiments.

FIG. 16 shows a front view of a blade for a flat blade tissue penetrating device tip according to some embodiments.

FIG. 17 shows a side view of a blade for a flat blade tissue penetrating device tip according to some embodiments.

FIG. 18 shows a perspective view of a flat blade tissue penetrating device tip according to some embodiments.

FIG. 19 shows a perspective view of a flat blade tissue penetrating device tip according to some embodiments.

FIG. 20 shows a perspective view of a flat blade tissue penetrating device tip according to some embodiments.

FIG. 21 shows a perspective view of a flat blade tissue penetrating device tip according to some embodiments.

FIG. 22 shows a perspective view of a flat blade tissue penetrating device tip according to some embodiments.

FIG. 23 shows a front view of an ablation instrument, a portion of which is shown by a cross-section view, according to some embodiments.

FIG. 24 shows a front view schematic of an ablation system and target tissue, including a catheter cross-section, according to some embodiments.

FIG. 25 shows a front transparent view of an antenna body according to some embodiments.

FIG. 26 shows a front view of a tissue penetrating device tip according to some embodiments.

### DETAILED DESCRIPTION

In the following description, numerous specific details are set forth in order to

provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring aspects of the disclosure.

References in the specification to one embodiment, an embodiment, an example embodiment, etc., indicate that the embodiment described may include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is within the knowledge of one skilled in the art to affect such feature, structure, or characteristic in connection with other embodiments whether or not explicitly described.

The following examples are illustrative, but not limiting, of the present disclosure. Other suitable modifications and adaptations of the variety of conditions and parameters normally encountered in the field, and which would be apparent to those skilled in the art, are within the spirit and scope of the disclosure.

As noted above, penetrating certain types of tissue, such as smaller tumors or other tough tissue, may present challenges because smaller tumors may be surrounded by more compliant tissue and may be easily displaced. If the tumor is to be ablated, it may be difficult to ensure appropriate placement of an ablation antenna (e.g., in the center of the tumor) when the tumor is unintentionally displaced.

The present disclosure relates to tissue penetrating device tips having one or more cutting edges. The device tips may be configured to penetrate tissue, such as small tumors (e.g., having a largest dimension of less than 3 cm), which may be cancerous or benign. The device tip may penetrate the tumor while maintaining accurate aim by minimizing displacement of the tumor and of the distal end of a catheter for delivering instruments with device tips, both of which may be supported by compliant surrounding tissue. A device (e.g., a medical instrument) having an appropriate tissue penetrating device tip may penetrate tissue using a lower penetration force, which may minimize displacement of the tissue being penetrated. The size of device tips and medical instruments described herein may be kept small in order to facilitate access to target anatomy. Manufacturing costs of device tips and medical instruments described herein may be kept low. In the case of an ablation instrument, the amount of metal used for the device tip may be kept at a minimum for improved high frequency electrical performance. Embodiments of the present disclosure provide for improved device tips for penetrating tissue.

The present disclosure provides for various structural and mechanical configurations for device tips used to penetrate tissue, and in some cases ablative instruments which penetrate tissue and deliver ablative energy. Penetrating tissue, such as the capsule of a tumor, may occur based on a combination of cutting and stretching the tissue. Increased cutting capability may reduce the force necessary to penetrate the tissue. However, an increase in stretching requirement may increase the necessary tissue penetration force. For example, a conical pointed instrument tip may do very little, if any, tissue cutting, but rather may stretch and tear the tissue as the instrument penetrates the tissue. Thus, a conical pointed instrument tip may require a high tissue penetration force. Various designs for medical instrument or device tips with improved ability to penetrate tissue, including tough tissue such as some types of tumors, will be described below. Device tips described herein may be used to penetrate any type of tissue, including tumor tissue, depending on tissue toughness and location of target anatomy (e.g., tortuosity of anatomy for a minimally invasive delivery of the medical instrument, anatomical space constraints for approaching a target, etc.).

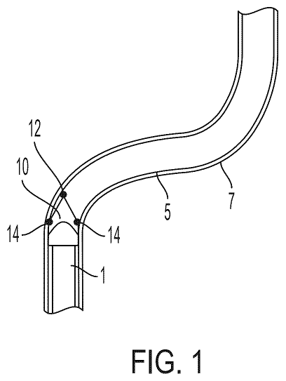

As shown in FIG. 1, a flexible elongate device 7 (e.g., a catheter) may be positioned within a patient anatomy, such as the lungs, intestine, ureter, kidney, and/or other patient anatomy. The flexible elongate device 7 may be inserted into the patient anatomy through a natural opening, such as the mouth, nose, cars, anus, urethra, or vagina of the patient, or through an artificial opening such as one created by a surgical incision. After the flexible elongate device 7 is positioned to access a target anatomy (e.g., anatomy having a tumor), an instrument 1 may be inserted through a lumen 5 of the flexible elongate device 7 to access the target anatomy. In some instances, an inside wall of the lumen 5 may define a tortuous path to the target anatomy. If not sized and shaped appropriately, an instrument 1 with a sharp tip or blade 10 may cut a portion of the inside wall of the lumen 5 as the instrument 1 goes around a sharp bend of the tortuous path. For example, instrument blade 10 may cut the inside wall of lumen 5 at a tip contact point 12 and/or at a side contact point 14. The width of blade 10′s cutting edges may affect the likelihood that blade 10 will cut the inside wall of lumen 5.

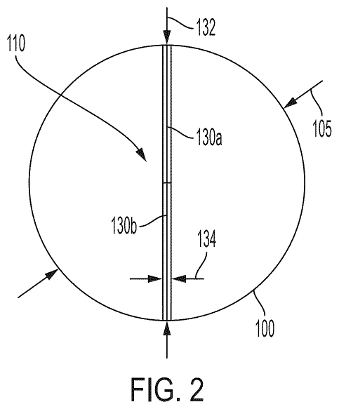

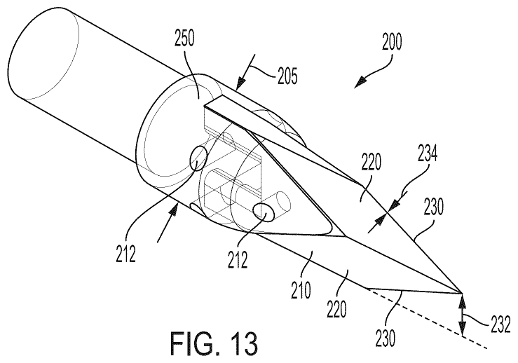

FIG. 2 shows an illustrative tissue penetrating device tip 100 from a top schematic view. For example, FIG. 2 may be a top schematic view of the tissue penetrating device tip 200 shown in FIG. 13, which will be described in further detail below. As shown in FIG. 2, device tip 100 may have a cross-sectional diameter 105. The cross-sectional diameter 105 may be selected so that the device tip 100 fits within a lumen of a catheter used to deliver an instrument with the device tip 100. For example, cross-sectional diameter 105 may be less than approximately 5 mm, less than approximately 3 mm, or less than approximately 2 mm. The device tip 100 may also include a blade 110 having a pair of intersecting cutting edges 130a and 130b. The blade 110 may have a width 132 and a thickness 134. As used herein, the terms thickness, thick, and thin may refer to the smaller transverse dimension of a blade (e.g., a cutting edge of a blade), and the terms width, wide, and narrow may refer to the larger transverse dimension of the blade (e.g., a cutting edge of a blade). With a width 132, the device tip 100 may make a cut of approximately width 132 when the blade 110 is fully inserted into the tissue.

As explained above, tissue penetrating device tips having one or more cutting edges may allow penetration of tissue using a lower penetration force and reduce the likelihood that the tissue (e.g., a tumor) is displaced by the device tip. A stretch ratio for a device tip may be used to indicate the perimeter of an opening in the tissue after cutting and stretching compared to the perimeter of the opening after cutting only by a blade of the device tip. A higher stretch ratio may indicate more tissue stretching, and a higher penetration force may be needed to penetrate the tissue. A lower stretch ratio may indicate less tissue stretching and/or more tissue cutting, and a lower penetration force may be needed to penetrate the tissue.

For the device tip 100 of FIG. 2, the perimeter of the opening may correspond to the circumference of the device tip 100 at the cross-sectional diameter 105, which may be the maximum tip diameter of the device tip. Thus the perimeter may be π times the diameter 105. The cut by device tip 100 may correspond to the perimeter of a slit cut by blade 110 of the device tip 100. With the pair of cutting edges 130a and 130b, as shown in FIG. 2, the perimeter of cut tissue is approximately two times the length of the slit cut by the blade 110 or two times the width 132 (e.g., assuming that the thickness 134 is much smaller than the width 132). As shown in FIG. 2, the blade 134 extends from one end of the device tip 100 to the other end, and the amount of tissue cut may be approximately two times the diameter 105. Accordingly, the stretch ratio of the device tip 100 may be approximately 90 /2 (e.g., (π*d₁₀₅)/(2*d₁₀₅)).

While FIG. 2 shows a blade width 132 equal to the cross-sectional diameter 105 of the device tip 100, width 132 may be less than the cross-sectional diameter 105. In some examples, width 132 may be between 60% and 100% of cross-sectional diameter 105. For example, width 132 may be approximately 80% of cross-sectional diameter 105.

A greater width 132 of cutting edge(s) 130 may result in more cutting and thus reduce the stretch ratio. For example, when width 132 is equal to cross-sectional diameter 105, the stretch ratio may be approximately π/2 as explained above. When width 132 is smaller than cross-sectional diameter 105, the stretch ratio may increase. However, reducing the width 132 may be helpful to avoid an inside wall of a catheter being cut by cutting edge 130a and/or 130b as the instrument having the device tip 100 is navigated through the catheter. A device tip with a single cutting edge may result in a similar stretch ratio as the device tip 100 with two cutting edges 130a and 130b that are aligned with each other because the widths of the two cutting edges together (e.g., the total width of the blade) equals width 132.

Cutting more tissue, such as by increasing the perimeter of cut tissue, may reduce the stretch ratio and/or the required penetration force. Cutting more tissue can be achieved by increasing the number of cutting edges of an instrument tip. However, there may be a threshold number of cutting edges beyond which the required penetration force may start to increase. An increase in the number of edges could also result in an increase in manufacturing costs.

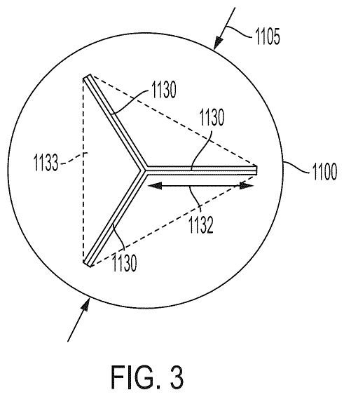

FIG. 3 shows an illustrative tissue penetrating device tip 1100 from a top schematic view. Device tip 1100 may have a cross-sectional diameter 1105. Device tip 1100 may have three cutting edges 1130, each having a width 1132. In some embodiments, width 1132 of each cutting edge 1130 may be equal to each other. With the arrangement of three cutting edges 1130 shown in FIG. 3, the perimeter 1133 of cut tissue may be approximately a triangular shape, represented by the dotted line in FIG. 3. The amount of tissue stretch may correspond to the circumference of the device tip 1100 at the cross-sectional diameter 1105. Accordingly, the stretch ratio for the device tip 1100 may be approximately the circumference of the device tip 1100 (e.g., π*d₁₁₀₅) divided by the length of the triangular perimeter 1133.

In some embodiments, cross-sectional diameter 1105 may be less than approximately 5 mm, less than approximately 3 mm, or less than approximately 2 mm. In some embodiments, width 1132 may be less than half the cross-sectional diameter 1105 (e.g., as shown in FIG. 3), or width 1132 may be equal to half the cross-sectional diameter 1105. In some examples, width 1132 may be between 60% and 100% of half the cross-sectional diameter 1105 (or 30% to 50% of the cross-sectional diameter 1105). For example, width 1132 may be approximately 80% of half the cross-sectional diameter 1105 (or 40% of the cross-sectional diameter 1105).

A greater width 1132 of each cutting edge 1130 may increase the amount of tissue cut and thus reduce the stretch ratio. However, reducing the width 1132 may be helpful in some instances to avoid the catheter lumen inside wall being cut by the point or an outer corner of cutting edge 1130, as shown by tip contact point 12 and side contact point(s) 14 of FIG. 1.

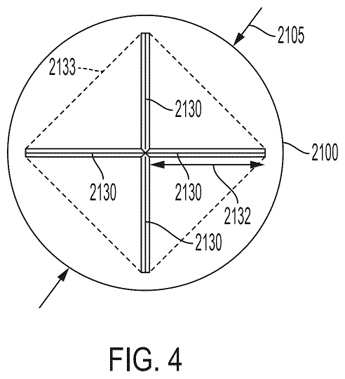

FIG. 4 shows an illustrative tissue penetrating device tip 2100 from a top schematic view. Device tip 2100 may have a cross-sectional diameter 2105. Device tip 2100 may have four cutting edges 2130, each having a width 2132. In some embodiments, width 2132 of each cutting edge 2130 may be equal to each other. With the arrangement of four cutting edges 2130 shown in FIG. 4, the perimeter 2133 of cut tissue may be approximately a rectangular or square shape, represented by the dotted line in FIG. 4. The amount of tissue stretch may correspond to the circumference of the device tip 2100 at the cross-sectional diameter 2105. Accordingly, the stretch ratio for the device tip 1100 may be approximately the circumference of the device tip 2100 (e.g., π*d₂₁₀₅) divided by the length of the rectangular perimeter 2133.

In some embodiments, cross-sectional diameter 2105 may be less than approximately 5 mm, less than approximately 3 mm, or less than approximately 2 mm. In some embodiments, width 2132 may be less than half the cross-sectional diameter 2105 (e.g., as shown in FIG. 4), or width 2132 can be equal to half the cross-sectional diameter 2105. In some examples, width 2132 may be between 60% and 100% of half the cross-sectional diameter 2105 (or 30% to 50% of the cross-sectional diameter 2105). For example, width 2132 may be approximately 80% of half the cross-sectional diameter 2105 (or 40% of the cross-sectional diameter 2105).

A greater width 2132 of each cutting edge 2130 may increase the amount of tissue cut and thus reduce the stretch ratio. However, reducing the width 2132 may be desirable in some instances to avoid the catheter being cut by the point or the outer corner of cutting edge 2130 as shown by tip contact point 12 and side contact point(s) 14 of FIG. 1.

As explained above, previous designs used cone point tips to penetrate tissue (e.g., tumor capsules). Because the cone point provides all stretch and no cut, it may have a very high stretch ratio in which stretching or tearing accounts for the entire slit expansion from the initial contact between the cone tip and the tissue to the fully stretched hole in the tissue. A tip that relies on all or excessive stretch or tearing and less or no cutting may continue to require higher penetration forces as the tip progresses into the tumor. In contrast, a device tip with one or more cutting edges (e.g., device tips 100, 1100, 2100 discussed above) may provide a much lower stretch ratio than a cone point tip, thus reducing the required penetration force. The slit, triangle or quadrangle polygon enclosing radial cuts formed by a number of cutting edges (e.g., 1 or 2, 3, or 4) extending from the center point of the device tip to its perimeter may be compared to the circumference of the device tip to determine the stretch ratio. Device tips may have more than four cutting edges, such as five cutting edges, six cutting edges, eight cutting edges, or other numbers of cutting edges. The perimeter of tissue cut by these device tips may be approximately a bounding polygon connecting the vertices or outer ends of the cutting edges. With more cutting edges, the length around that bounding polygon (e.g., the perimeter) may more closely approach a circular shape. And with wide cutting edges (e.g., cutting edges approaching the outside diameter of the device tip), the perimeter cut may approach the circumference of the device tip and the stretch ratio may approach 1 in these examples.

In addition to poor stretch ratio, previous designs (such as a three-edge flat-faced trocar tip) had poor performance due in part to the edge machining, which may have failed to create a keen edge. Keenness may refer to the thickness of the actual edge where faces of a cutting edge meet. Keenness may be measured using a scanning electron microscope (SEM). A thinner cutting edge may result in a keener cutting edge. For example, a trocar shape that is machined of polyether ether ketone (PEEK) plastic material might not allow as keen an edge as a metal material. Sharpness of cutting edges may also contribute to the cutting performance of a device tip. The term sharpness may refer to the dihedral angle between tip faces where the tip faces meet. A smaller dihedral angle may result in a sharper cutting edge. A traditional trocar-style flat-faced tip having (e.g.) a 71-degree dihedral angle between faces may have poor cutting performance.

In improved designs for minimally invasive applications, keenness and sharpness may be optimized for penetration of tissue with a lower penetration force (e.g., as low a penetration force as possible), while maintaining a small device tip size (including device tip diameter and cutting edge width). For example, the device tip may be made small enough to be accommodated for delivery by small lumen diameter catheters. Keenness may approach that of scalpels and shaving razors. For example, the keenness of the instrument tip may be approximately 0.1 μm=100 nm (0.000004″).

The sharpness may be as high as practical, which equates to a smaller dihedral angle, for the available space and tip geometry, while not being so high that the cutting edge is no longer durable and/or is too flexible that it bends under expected cutting forces or foreseeable accidentally applied forces against hard objects. In some embodiments, the dihedral angle of cutting edges described herein may be less than approximately 50 degrees. For example, the dihedral angle may be in the range of approximately 15 to approximately 40 degrees. In some embodiments, the dihedral angle may be in the range of approximately 25 to approximately 35 degrees. In some embodiments, the dihedral angle may be approximately 27 degrees.

In determining the optimal design for tissue penetrating device tips, considerations include low penetration force performance limit, manufacturing cost, and size. When a device tip is being integrated with an ablation device (which may have a microwave antenna), another consideration may be the desired reduction of the amount of metal to satisfy high frequency electrical performance goals of the antenna. Both the mass of metal and the axial length or extension of the blade due to sharpened face size are reduced by thinner blade material for a given dihedral angle. Reducing the axial extension of the blade may enable the blade to navigate a tighter radius bend in a catheter lumen without a point of the blade (e.g., the point 12 in FIG. 1) cutting into an inside wall of the catheter lumen (e.g., the lumen inside wall 5 in FIG. 1).

Although much of the present disclosure describes device tips for penetrating tumors, the device tips described herein may be used for penetrating other types of tissue. Moreover, the device tips may be used in both medical applications and non-medical applications.

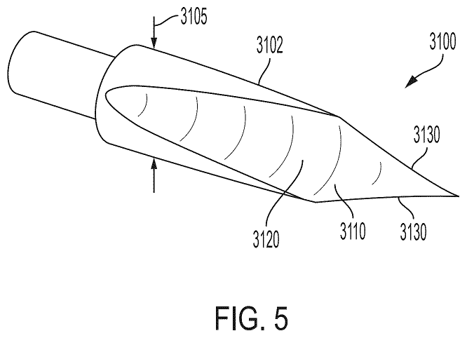

FIGS. 5-8 show an illustrative tissue penetrating device tip 3100 from different perspectives. Device tip 3100 may comprise a body portion 3102. In some embodiments, device tip 3100 may be sized to fit inside a catheter's lumen. For example, the body portion 3102 of device tip 3100 may have a cross-sectional diameter 3105 that is smaller than the inner diameter of a catheter's lumen. In some embodiments, device tip 3100 may have a cross-sectional diameter 3105 of less than approximately 5 mm. Specifically, a proximal portion of the body portion 3102 may have a diameter 3105 of less than approximately 5 mm. For example, device tip 3100 may have a cross-sectional diameter 3105 of approximately 4 mm, approximately 3 mm, approximately 2 mm, or approximately 1 mm. In some embodiments, device tip 3100 may have a cross-sectional diameter 3105 of less than approximately 3 mm, or less than approximately 2 mm.

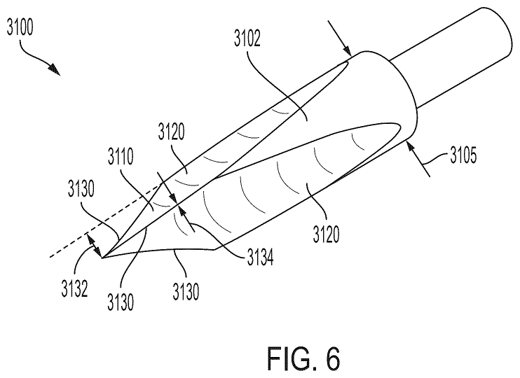

As shown in FIG. 5, device tip 3100 may comprise a blade 3110. Blade 3110 may be arranged distal to the proximal portion of the body 3102. Blade 3110 may have two or more tip faces 3120, which may form one or more cutting edges 3130. Cutting edges 3130 are located where two tip faces 3120 meet. In some embodiments, as shown, for example, in FIGS. 5-7, blade 3110 comprises three tip faces 3120 and three cutting edges 3130. As used herein, flat or curved surfaces may be referred to as faces or surfaces. The straight or curved line features where two surfaces meet may be referred to as edges. Corners may be the intersection of three or more surfaces and the edges they define at their paired intersections. As shown in FIGS. 6-8, each cutting edge 3130 has a width 3132, a thickness 3134, and a dihedral angle 3125.

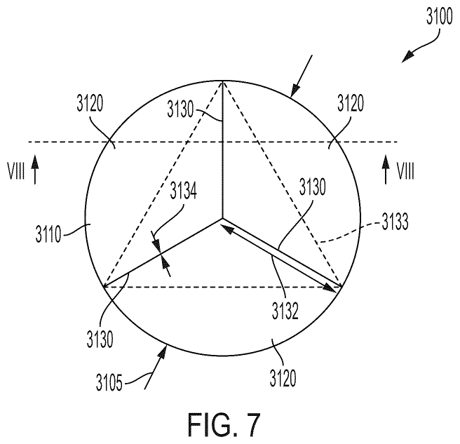

The width 3132 is shown in FIGS. 6 and 7. The width 3132 of each cutting edge 3130 may contribute to the overall width of blade 3110. As discussed above, in some embodiments, the width 3132 of each cutting edge 3130 is narrow enough that it does not cut a catheter lumen liner, particularly in a tight bend, but wide enough to produce a cut tissue perimeter that reduces the stretch ratio and the penetration force. Thus, width 3132 of individual cutting edge 3130 may contribute to the desired stretch ratio. In some embodiments, each cutting edge 3130 may have a width 3132 of between approximately 0.5 mm and approximately 2.5 mm.

Because device tip 3100 has three cutting edges (like device tip 1100 shown in FIG. 3), the discussion with respect to device tip 1100 applies to device tip 3100. For example and with reference to FIG. 7, the amount of tissue stretch caused by device tip 3100 may correspond to the circumference of the device tip 3100 at the cross-sectional diameter 3105 (e.g., π times the cross-sectional diameter 3105). The amount of tissue cut may be approximately the length of the triangular perimeter 3133 formed by the ends of each of the cutting edges 3130. Accordingly, the stretch ratio for the device tip 3100 may be approximately the circumference of the device tip 3100 (e.g., π*d₃₁₀₅) divided by the length of the perimeter 3133.

The thickness 3134 of a cutting edge 3130 is shown in FIGS. 6 and 7. The thickness 3134 may also contribute to reducing the required penetration force. Specifically, a thinner cutting edge 3130 may result in higher keenness, which may reduce required penetration force. In some embodiments, cutting edge 3130 has a thickness of less than approximately 1 micron. In some embodiments, cutting edge 3130 has a thickness of less than approximately 0.1 micron.

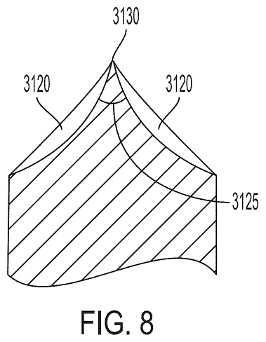

With reference to FIG. 6, the dihedral angle of the cutting edge 3130 may be defined by a cutting plane perpendicular to the cutting edge 3130 (or a cutting plane perpendicular to the cutting edge at a point along the cutting edge 3130 if the dihedral angle of the cutting edge 3130 varies along its length). The dihedral angle 3125 of a cutting edge 3130 is shown in FIG. 8. The dihedral angle 3125 may contribute to reducing the required penetration force. Specifically, a smaller dihedral angle 3125 results in higher sharpness, which may reduce required penetration force.

In some embodiments, device tip 3100 can be a hollow ground tip. Hollow ground tips may be manufactured by grinding multiple various shaped faces 3120 from one piece. For example, a toroidal or donut-shaped outer surface grinding wheel may be used to grind the tip faces 3120. In some embodiments, hollow ground tips 3100 may reduce the required penetration force compared to previous designs by 2× (or one half the penetration force of previous designs) due to the increased sharpness from grinding away portions of the blade to form a concave surface decreasing the dihedral angle while increasing the sharpness and due to the decreased stretch ratio. Thus, for device tip 3100, the shape of the tip faces 3120 and the angle at which they are ground may help achieve a particular level of sharpness. In some embodiments, the tip faces 3120 can be ground such that they curve inwards and are concave. When the tip faces 3120 are concave, the dihedral angle 3125 of the cutting edge 3130 formed by two tip faces 3120 may vary along the cutting edge 3130.

Hollow grinding may reduce the dihedral angle 3125 between adjacent tip faces 3120. The dihedral angle 3125 between adjacent tip faces 3120 may be less than approximately 50 degrees. In some embodiments, the dihedral angle 3125 between adjacent tip faces 3120 may be between approximately 25 and approximately 35 degrees. For example, the dihedral angle 3125 between each adjacent tip face 3120 of a three-sided hollow ground tip 3100 may be approximately 27 degrees. In contrast, a conventional trocar with three flat faces may have a dihedral angle of 71 degrees. Thus, the advantage of the 3-sided hollow ground geometry is that, unlike a conventional trocar with 3 flat faces, the angle 3125 between the hollow ground faces 3120 where they meet at cutting edge 3130 can be much sharper than a conventional trocar (e.g., 27 degrees vs. 71 degrees). This 3-sided hollow ground geometry creates a better cutting edge 3130 when properly honed, thus reducing the force needed to penetrate tough tissue, such as tumors.

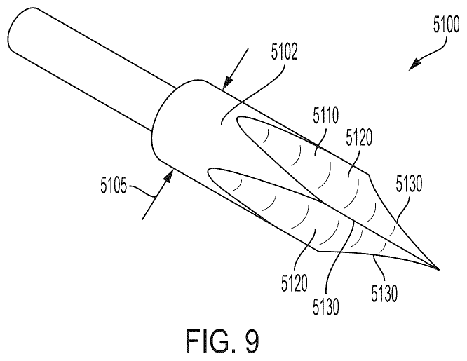

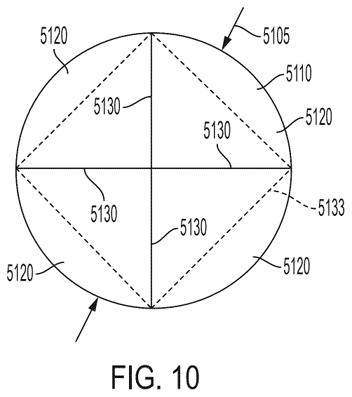

While a 3-sided hollow ground tip 3100 with three tip faces 3120 was described with reference to FIGS. 5-8, other hollow ground tips may have more or fewer tip faces. FIGS. 9 and 10 illustrate a 4-sided device tip 5100 comprising a body portion 5102. The body portion 5102 may have a cross-sectional diameter 5105. Similar to device tip 3100, device tip 5100 may be hollow ground. Device tip 5100 may comprise a blade 5110 with four tip faces 5120 and four cutting edges 5130. Because device tip 5100 has four cutting edges (like device tip 2100 shown in FIG. 4), the discussion with respect to device tip 2100 applies to device tip 5100. For example and with reference to FIG. 10, the amount of tissue stretch caused by device tip 5100 may correspond to the circumference of the device tip 5100 at the cross-sectional diameter 5105 (e.g., π times the cross-sectional diameter 5105). The amount of tissue cut may be approximately the length of the rectangular perimeter 5133 formed by the ends of each of the cutting edges 5130. Accordingly, the stretch ratio for the device tip 5100 may be approximately the circumference of the device tip 5100 (e.g., π*d₅₁₀₅) divided by the length of perimeter 5133.

In some embodiments, a device tip may include more than four cutting edges (and more than four tip faces). As discussed above, increasing the number of cutting edges may increase the perimeter of the cut tissue, which may reduce the stretch ratio and reduce the required penetrating force.

In some embodiments, to achieve the desired keenness for cutting edges 130, 1130, 2130, 3130, 5130, the device tips 100, 1100, 2100, 3100, 5100 may be made of a hardened metal. In some embodiments, device tips 100, 1100, 2100, 3100, 5100 may achieve a stretch ratio of approximately less than 5 or approximately less than 3.

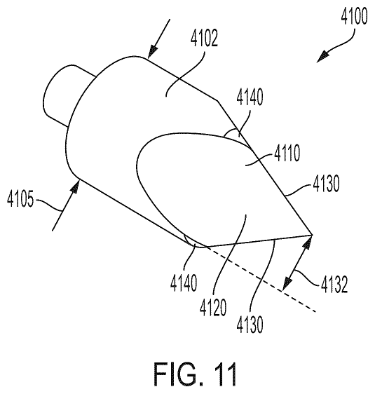

In another example, as shown in FIG. 11, a device tip 4100 can be manufactured from grinding an existing trocar. Device tip 4100 may comprise a body portion 4102. The body portion 4102 of device tip 4100 may have a cross-sectional diameter 4105. The device tip 4100 may include a blade 4110 with two or more faces 4120 and one or more cutting edges 4130. As noted above, an existing trocar may have an excessive dihedral angle between faces. Thus, tip faces 4120 may be formed by grinding an existing trocar to create the device tip 4100 having smaller dihedral angles. In some embodiments, the device tip 4100 can include rounded or flat outer corners 4140 to protect a catheter working lumen liner during delivery of the ground tip 4100 through the working lumen, particularly when being delivered through tight radial bends. In some embodiments, the working lumen of the catheter may have an inner diameter of less than 5 mm. Other device tips described herein (e.g., device tips 100, 1100, 2100, 3100, 5100 described above, or device tips 200, 1200, 2200, 3200, 4200, 5200 described below) may similarly include one or more rounded or flat outer corners 4140.

Cutting edges 4130 of blade 4110 have a width 4132, a thickness, and a dihedral angle. With rounded or flat outer corners 4140, width 4132 of cutting edge 4130 is less than diameter 4105 of device tip 4100. The discussion above regarding width 3132, thickness 3134, and dihedral angle 3125 of device tip 3100 (including various dimensions) also applies to width 4132, as well as cutting edge's 4130′s thickness and dihedral angle.

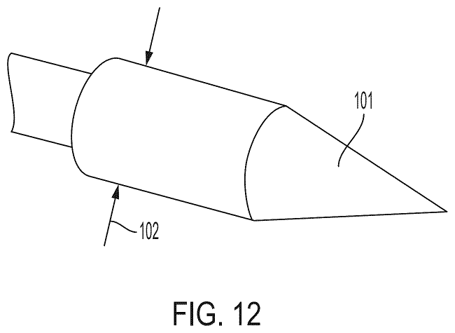

In another example, a ground tip can be formed starting with an integral cone 101, as shown in FIG. 12. For example, cone 101 may be provided for a device tip with diameter 102. However, a conical pointed tip does not cut, but rather stretches and tears tissue. Thus, faces (e.g., concave faces) may be ground into cone 101 to form cutting edges with widths, thicknesses, and dihedral angles similar to those described above. In some embodiments, two or more tip faces 3120 or 5120 may be created by grinding into cone 101, thus forming a ground tip with cutting edges, similar to device tip 3100 or 5100, for example.

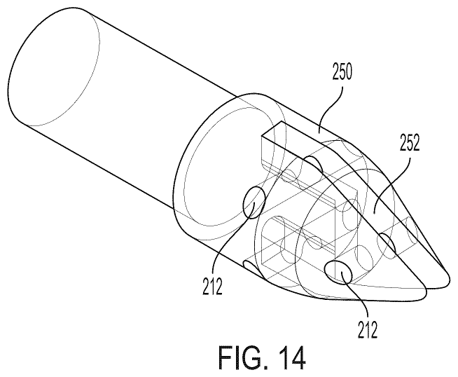

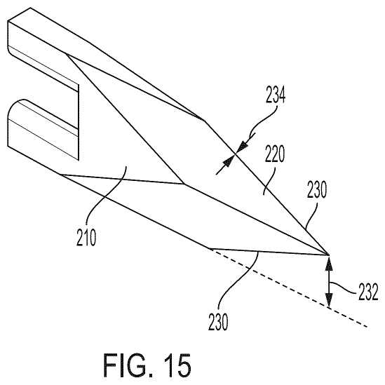

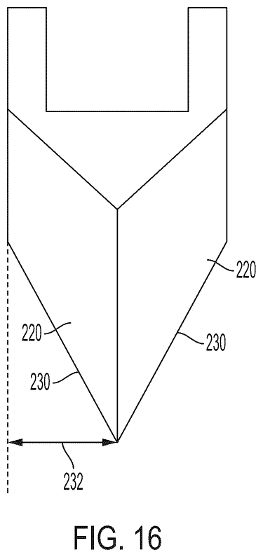

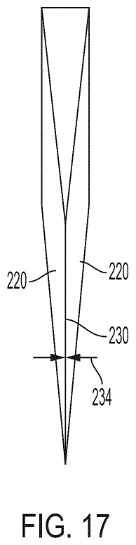

FIG. 13 shows an illustrative tissue penetrating device tip 200. In some embodiments, device tip 200 is a flat blade tip. Device tip 200 may comprise a blade 210, such as a sloping flat cutting blade, partially disposed or embedded within a body portion 250 of the device tip 200. The body portion 250 may comprise a cone shaped body 250, such as a dilating cone. The body portion 250 may have a cylindrical portion having a cross-sectional diameter 205. Cone shaped body 250 is shown in FIG. 14, and blade 210 is shown in FIGS. 15-17. Flat blade tip 200 may be sized to fit inside a catheter's lumen, and the cross-sectional diameter 205 may depend on the size of the catheter lumen through which tip 200 is to pass. Thus, the cross-sectional diameter 205 of flat blade tip 200 may be smaller than the inner diameter of a catheter's lumen. In some embodiments, flat blade tip 200 may have a cross-sectional diameter 205 of less than approximately 5 mm. For example, flat blade tip 200 may have a cross-sectional diameter 205 of approximately 4 mm, approximately 3 mm, approximately 2 mm, or approximately 1 mm. In some embodiments, flat blade tip 200 may have a cross-sectional diameter 205 of less than approximately 3 mm or less than approximately 2 mm.

Cone shaped body 250 may include a slot 252, and the blade 210 can be inserted into the slot 252 within the cone shaped body 250. Cone shaped body 250 may include holes 212 on each side for receiving a pin, screw, glue, or other fastener to secure blade 210 within a slot of cone shaped body 250. For example, blade 210 may be fixed in place within slot 252 with an adhesive which may be applied before assembly or after assembly through holes 212 and/or by capillary action at the edge of slot 252 where blade 210 emerges.

As shown in FIG. 13, the flat blade tip 200 comprises two cutting edges 230. A flat blade tip 200 with two cutting edges 230 may have an arrowhead shape. This configuration may cut a single slit in the tissue. The cutting edges 230 of blade 210 may each have a width 232, a thickness 234, and a dihedral angle (not shown). The dihedral angle of a cutting edge 230 may be defined by a cutting plane perpendicular to the cutting edge 230. The discussion of FIG. 6 above regarding width 3132, thickness 3134, and dihedral angle 3125 of device tip 3100 (including various dimensions) also applies to the width 232, the thickness 234, and the dihedral angle of a cutting edge 230 of flat blade tip 200. For example, the width 232 of one cutting edge 230 may be half the cross-sectional diameter 205 of the body portion 250. In other examples, the width 232 may be less than half the cross-sectional diameter, such as between 30% and 50% of the cross-sectional diameter 205 or 40% of the cross-sectional diameter. A wider blade 210 (in the width 232 direction) increases the length of the cut tissue. A thinner blade 210 (in the thickness 234 direction) allows smaller tip faces 220 at a given dihedral angle 225 and can result in a shorter tip 200 that can be wider (in the width 232 direction) without cutting a catheter liner in tight radius bends, as discussed above with respect to FIG. 1.

The thickness 234 of a cutting edge 230 may be approximately 0.1 μm, and the dihedral angle of the cutting edge 230 may be less than approximately 50 degrees. For example, the dihedral angle may be in the range of approximately 15 to approximately 40 degrees. In some embodiments, the dihedral angle may be in the range of approximately 25 to approximately 35 degrees. In some embodiments, the dihedral angle may be approximately 27 degrees. The dihedral angle of cutting edge 230 may be formed in the same way as a scalpel blade or shaving razor (e.g., by direct control of the dihedral angle along a straight cutting edge 230, or curved cutting edge, without the need for hollow grinding). By avoiding hollow grinding, flat blade tips 200 may have a manufacturing advantage over a hollow ground tip.

Because device tip 200 has two cutting edges 230 (similar to device tip 100 shown in FIG. 2), the discussion with respect to device tip 100 applies to device tip 200. For example and with reference to FIG. 2, the amount of tissue stretch caused by device tip 200 may be similar to the amount of tissue stretch caused by device tip 100. Thus the amount of tissue stretch may correspond to the circumference of the device tip 100 or 200 at the cross-sectional diameter 105 or 205 (e.g., π times the cross-sectional diameter 105 or 205). The amount of tissue cut may be approximately the length of the perimeter formed by the cutting edges 130a and 130b or cutting edges 230, which may be approximately two times the width 132 or four times the width 232. Accordingly, the stretch ratio for the device tip 100 or 200 may be approximately π/2 (e.g., (π*d₁₀₅)/(2*d₁₀₅) for device tip 100 or (π*d₂₀₅)/(4*w₂₃₂) for device tip 200). Device tip 200 may reduce the required penetration force compared to previous designs by 4× (or one quarter of the penetration force of previous designs) due to the increased sharpness of the cutting edge(s) and due to the decreased stretch ratio compared to previous designs.

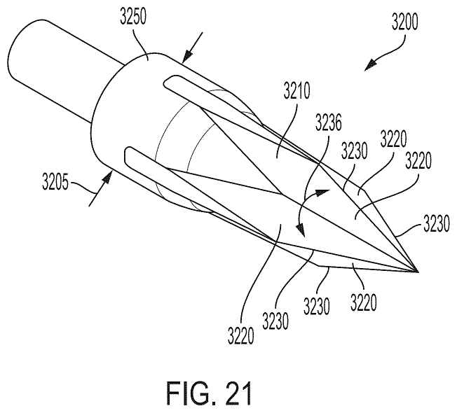

While one blade 210 with two cutting edges 230 is shown in FIG. 13, a device tip may include any number of blades 210, and blades 210 can include any number of faces 220 at various dihedral angles, thicknesses 234, and widths 232 creating sharp edges 230 as necessary. For example and as discussed further below, FIG. 21 illustrates a device tip 3200 with two blades 3210 and four cutting edges 3230. In embodiments with multiple blades 3210 and/or cutting edges 3230, the blades 3210 and/or cutting edges 3230 may be at equal angular spacing 3236 from each other or at varied spacing from each other depending on applications.

More blade edges may increase the perimeter or length of the tissue cut, which may reduce the stretch ratio and reduce the required penetrating force. As previously explained, the amount of tissue cut for one or two cutting edges may be approximately two times the slit length 132 shown in FIG. 2, for three cutting edges may be approximately the triangular perimeter 1133 shown in FIG. 3, and for four cutting edges may be approximately the rectangular perimeter 2133 shown in FIG. 4. After some number of cutting edges, as the number of radial cuts being made increases, the cutting induced penetration force increases. The increase may be proportional to the number of radial cutting edges. Thus, there may be a tradeoff between increasing the number of cutting edges to reduce stretching while increasing the cutting induced component of penetration force beyond the reduction in stretching induced penetration force. Moreover, additional cutting edges could also increase manufacturing cost.

Flat blade tips, such as device tip 200 in FIG. 13, may have less metal than hollow ground tips. When an electrically conductive tip is integrated into an ablation device, the electrically conductive tip could distort the microwave ablation field shape and uniformity. It could also result in excessive tip self-heating that can cause tissue dehydration and charring near the antenna prior to the desired cell death in the tumor volume to satisfy desired margins. Thus, in some embodiments, cone shaped body 250 may be made of plastic to reduce the amount of metal in the flat blade tip 200. Flat blade tip 200 may be designed to withstand elevated temperatures during an ablation operation, and cone shaped body 250 may be made of high temperature plastic in some embodiments. For example, cone shaped body 250 may be a polyaryl ether ketone (PAEK), or polyether ether ketone (PEEK), or liquid crystal polymer (LCP), or Radel® polyphenylsulfone, Amodel® polyphthalamide, or other high temperature-resistant plastics. These materials for cone shaped body 250 can withstand high temperatures, yet might not have self-heating or electromagnetic field shape effects. By using such materials for cone shaped body 250, volume of metal may be limited to blade 210.

A tissue compatible lubricious coating may be incorporated on the cutting blade 210 and/or cone shaped body 250. The lubricant coating may be pre-applied on the cutting blade 210 and/or cone shaped body 250, or the lubricant coating may be applied at the time of use of flat blade tip 200. In some embodiments, the lubricant coating may be grease or oil (e.g., silicone oil, white mineral oil, etc.). In some embodiments, the lubricant coating may be parylene, polytetrafluoroethylene (PTFE), Hydak® hydrophilic coatings from Biocoat Incorporated, or another deposited thin coating. In embodiments in which blade 210 is non-metal, a lubricant may be compounded into a material of the cutting blade 210 and/or cone shaped body 250. For example, PTFE and/or silicone oil may be compounded into the plastic of cone shaped body 250. In some embodiments, combinations of the foregoing lubricants or other equivalent options may be used for flat blade tip 200. Any of the lubricants described with respect to flat blade tips 200 may be used with other tips described herein, such as tips 100, 1100, 2100, 3100, 4100, or 5100.

In some embodiments, a flat blade tip may comprise one cutting edge 230, thus forming a chisel-style blade. However, this chisel-style blade may have a greater tendency (when compared to flat blade tips 200 with two cutting edges 230) to cut a catheter lumen liner due to its protruding corner(s).

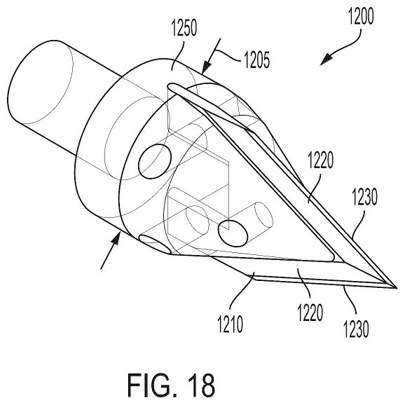

FIG. 18 shows an illustrative tissue penetrating device tip 1200. Device tip 1200 may be a flat blade tip. Flat blade tip 1200 may include a body portion 1250 (e.g., a cone shaped body portion) having a cross-sectional diameter 1205. The tip 1200 may include a blade 1210 with two or more faces 1220 and two cutting edges 1230. In some embodiments, blade 1210 may be shorter than blade 210 of FIGS. 13, 15, 16 and 17. For example, blade 1210 extends distally out of cone shaped body 1250 by a shorter distance than blade 210 extends out of cone shaped body 250. Having a shorter blade 1210 may help device tip 1200 traverse the lumen of a catheter without snagging or cutting the inner wall of the catheter lumen. This short length may be provided while still maintaining low tumor penetration forces. In some embodiments, blade 1210 may extend distally out of cone shaped body 1250 by less than approximately 1 mm. In some embodiments, blade 1210 may extend distally out of cone shaped body 1250 by less than approximately 0.5 mm. Cutting edges 1230 may have a width, thickness, and/or dihedral angle similar to cutting edges 230 shown in FIGS. 13 and 15-17 or cutting edges 3130 shown in FIGS. 5-8. Thus, the discussion above regarding width 3132, thickness 3134, and dihedral angle 3125 of device tip 3100 (including various dimensions) and width 232, thickness 234, and dihedral angle of device tip 200 (including various dimensions) also applies to flat blade tip 1200.

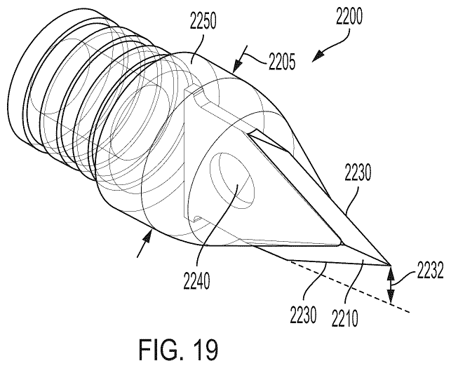

In some embodiments, as shown in FIG. 19, for example, a device tip 2200 comprises a body portion 2250 (e.g., a cone shaped body) that is overmolded onto a blade 2210. For example, a dilating cone 2250 of a high temperature thermoplastic may be overmolded onto a blade 2210. Thus, blade 2210 with its cutting edges 2230 may be embedded in cone shaped body 2250. In some embodiments, blade 2210 may include a hole 2240 to strengthen the connection between cone shaped body 2250 and blade 2210. During molding, the plastic may fill in hole 2240 and solidify such that a portion of cone shaped body 2250 surrounds and passes through blade 2210. An advantage to thin cutting edges 2230 embedded in a dilating cone 2250 (e.g., by overmolding a dilating cone 2250 of a high temperature thermoplastic onto a blade 2210) is that the device tip 2200 can be made shorter and can traverse a working lumen of a catheter without snagging or cutting the inner wall of the working lumen.

Because blade 2210 is embedded within cone shaped body 2250, the overall width of the blade 2210 may be less than a diameter 2205 of the cone shaped body 2250. Thus, as shown in FIG. 19, each cutting edge 2230 may have a width 2232 that is less than half of diameter 2205, which may help device tip 2200 traverse a catheter lumen without snagging or cutting the inner wall of the catheter lumen. In some embodiments, cutting edge 2230 may have the same or similar characteristics (e.g., thickness, dihedral angle, width, etc.) as other cutting edges discussed above, such as cutting edge 3130, cutting edge 230, etc.

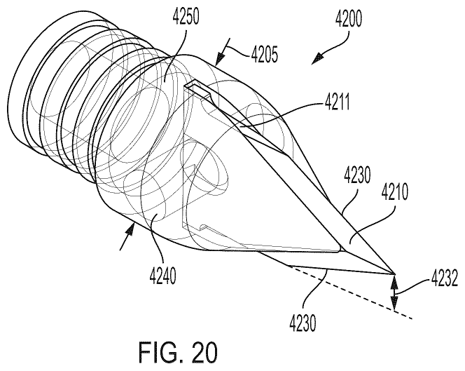

FIG. 20 shows a flat blade tip 4200 that comprises a body portion 4250 (e.g., a cone shaped body portion) and a blade 4210. Flat blade tip 4200 may be similar to flat blade tip 200, flat blade tip 1200, and flat blade tip 2200. For example, blade 4210 may have two cutting edges 4230. However, blade 4210 may have a side 4211 that is spaced inwards from the outer surface of cone shaped body 4250, as shown in FIG. 20. In some embodiments, blade 4210 has an overall width that may be less than a cross-sectional diameter 4205 of device tip 4200. Thus, cutting edge 4230 may have a width 4232 that is less than half of cross-sectional diameter 4205, which may help device tip 4200 traverse a catheter lumen without snagging or cutting the inner wall of the catheter lumen. Blade 4210 may have a similar inwardly disposed surface on an opposite side of blade 4210. In some embodiments, cutting edge 4230 may have the same or similar characteristics (e.g., thickness, dihedral angle, width etc.) as other cutting edges discussed above, such as cutting edge 3130, cutting edge 230, etc. In some embodiments, cone shaped body 4250 may include a hole 4240 extending from one side of body 4250 to the other side of the body 4250. The hole 4240 may be configured to receive a pin or other fastener that engages a slot or hole in a back edge of blade 4210 in order to hold blade 4210 in the slot of cone shaped body 4250.

Although blades 210, 1210, 2210, and 4200 each have two cutting edges 230, 1230, 2230, and 4230, some embodiments may include more than two cutting edges. For example, a flat blade tip may comprise three radial flat blades that form three radial cutting edges. In some embodiments, the three radial flat blades may have an equal angular spacing between adjacent blades. Thus, the angle between adjacent blades may be 120 degrees. The discussion with respect to device tip 1100 (which also has three cutting edges, as shown in FIG. 3) also applies to such a device tip.

FIG. 21 shows a flat blade tip 3200 with more than two cutting edges 3230. Flat blade tip 3200 has a cross-sectional diameter 3205 and may include a body portion 3250 (e.g., a cone shaped body) and one or more blades 3210. In some embodiments, flat blade tip 3200 comprises two intersecting flat blades 3210, thus forming four cutting edges 3230. In some embodiments, the angular spacing 3236 between adjacent cutting edges 3230 may be equal. Thus, the angle between each of the four cutting edges 3230 may be 90 degrees. In some embodiments, cutting edge 3230 may have the same or similar characteristics (e.g., thickness, dihedral angle, width, etc.) as other cutting edges discussed above, such as cutting edge 3130, cutting edge 230, etc.

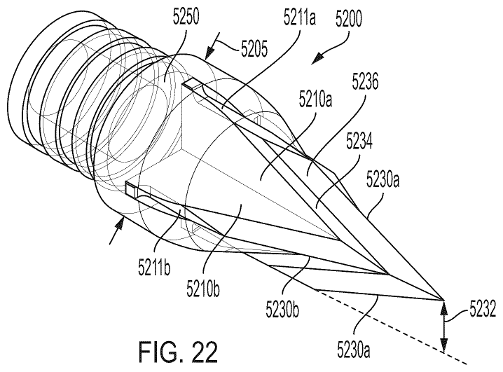

FIG. 22 shows a flat blade tip 5200 that comprises a body 5250 (e.g., a cone shaped body) and two intersecting blades 5210a and 5210b at right angles to each other and offset from each other in the axial direction. Flat blade tip 5200 may be similar to flat blade tip 3200. For example, each blade 5210a and 5210b may have two cutting edges 5230a and 5230b respectively, for a total of four cutting edges. However, blades 5210a and 5210b may have sides 521a and 5211b that are spaced inwards from the outer surface of cone shaped body 5250, as shown in FIG. 22. In some embodiments, each blade 5210a and 5210b may have an overall width that may be less than a cross-sectional diameter 5205 of device tip 5200. Thus, as shown in FIG. 22, each cutting edge 5230a and 5230b may have a width 5232 that is less than half of cross-sectional diameter 5205, which may help device tip 5200 traverse a catheter lumen without snagging or cutting the inner wall of the catheter lumen. The axial position of blade 5210a may be offset relative to the axial position of blade 5210b so that the distal end of the proximal blade 5210b converges to a flat side 5234 of the distal blade 5210a before reaching an angled face 5236 of the distal blade 5210a. This may prevent a gap that would occur if the intersecting blades 5210a and 5210b were at the same axial position. The gap may be eliminated by the axial positions shown in FIG. 22 (or other axial positions where the distal end of the proximal blade converges to a flat surface of the distal blade) so that tissue does not become snagged in a gap between the distal tips of the two blades. In some embodiments, cutting edge 5230 may have the same or similar characteristics (e.g., thickness, dihedral angle, width, etc.) as other cutting edges discussed above, such as cutting edge 3130, cutting edge 230, etc.

In some embodiments, flat blade tips (e.g., tips 200, 1200, 2200, 3200, 4200, 5200) may achieve a stretch ratio of less than approximately 5. In some embodiments, flat blade tips may achieve a stretch ratio of less than approximately 3. For example, flat blade tips may achieve a stretch ratio of approximately 1.5. In some embodiments, flat blade tips may achieve a stretch ratio of approximately 1.1.

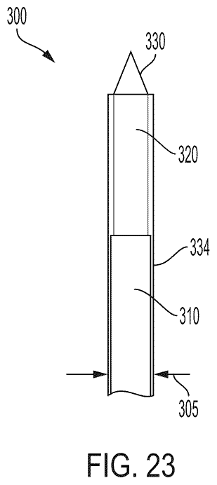

The hollow ground tips and flat blade tips discussed above may be used in various ablation systems (e.g., radiofrequency ablation systems, microwave ablation systems, etc.). FIG. 23 shows an ablation instrument 300 configured to penetrate tissue, such as a tumor, and ablate the tissue. In some embodiments, ablation instrument 300 comprises a microwave antenna assembly. The microwave antenna assembly can include a cable 310 (e.g., a coaxial cable), an antenna body 320 (e.g., a conductive antenna body), and an antenna tip 330. In some embodiments, the coaxial cable and antenna body may be disposed within an outer jacket 334. Antenna tip 330 may be any of the device tips discussed above (e.g., 100, 1100, 2100, 3100, 4100, 5100, 200, 1200, 2200, 3200, 4200, 5200). In some embodiments, the cable 310 may deliver current to the antenna body 320. The antenna body 320 may be coupled to the cable 310 and may be configured to deliver ablative energy to the target tissue. The dimensions of tip 330 may be any of the dimensions discussed above with respect to any of the tips disclosed herein. In some embodiments, a diameter 305 of the instrument 300 (e.g., including the coaxial cable 310, the antenna body 320, and the tip 330) may be less than approximately 5 mm, less than approximately 3 mm, or less than approximately 2 mm.

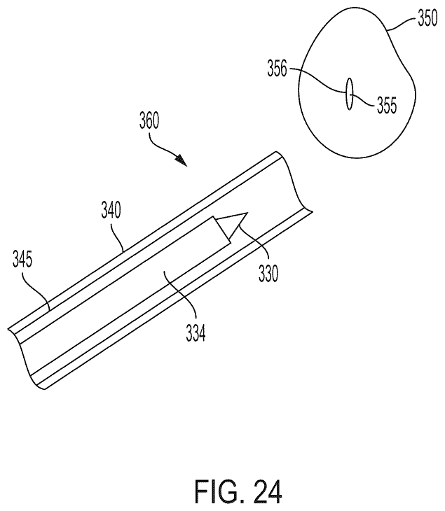

FIG. 24 shows an ablation system 360, which may include the ablation instrument 300 delivered through a catheter 340. Ablation instrument 300, including tip 330, may pass through an inner lumen 345 of the catheter 340 positioned to reach the target tissue 350. In some embodiments, catheter 340 (e.g., inner lumen 345) has an inner diameter of less than 5 mm. In some embodiments, catheter 340 (e.g., inner lumen 345) has an inner diameter of less than 2 mm. In some embodiments, catheter 340 may be navigated to target tissue 350, which may be a tumor or suspected tumor. In some scenarios, target tissue 350 may have a largest dimension of less than 3 cm. In other scenarios, target tissue 350 may be larger than 3 cm. As explained above, tip 330 may comprise one or more blades (e.g., any of the blades discussed above) that is configured to cut a slit 355 in the target tissue 350. The size of the slit 355 may depend on the size of the blades. In some embodiments, the slit 355 may have a perimeter 356 of at least approximately 1 mm. In some embodiments, the slit 355 may have a perimeter 356 of at least approximately 2 mm.

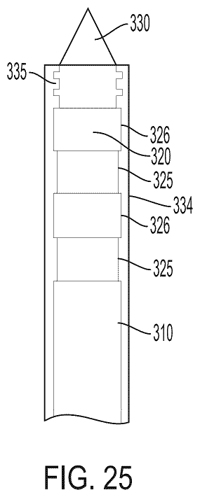

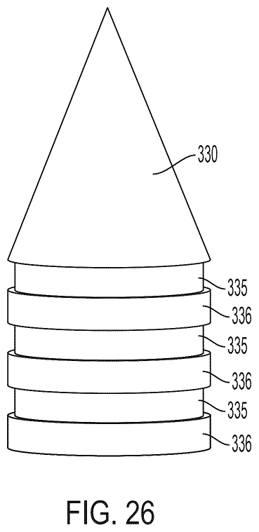

In some embodiments, the antenna body 320 comprises grooves 325, as shown, for example, in FIG. 25. Grooves 325 may facilitate coupling of antenna body 320 to outer jacket 334. For example, antenna body 320 could be grooved to allow for fluoropolymers (such as FEP) or another sealant to be melted around antenna body 320 and adhere to outer jacket 334 to create a water tight seal. In some embodiments, grooves 325 are exterior grooves. Grooves 325 of antenna body 320 may be separated by protrusions 326. As shown in FIGS. 25 and 26, grooves 335 can be provided on a portion of the tip 330 which can aid in attaching the tip 330 to the outer jacket 334. Accordingly, outer jacket 334 forms a layer coupling antenna body 320 to tip 330. Grooves 335 may be separated from one another by protrusions 336. Various devices and methods for attaching tips to antenna structures are described in US patent application docket #ISRG13570/US filed Oct. 31, 2019, disclosing “Coiled Antennas with Fluid Cooling”, which is incorporated herein by reference in its entirety.

In some alternative embodiments, antenna body 320 may be shaped so as to mate with tip 330 in order to secure the antenna body 320 to the tip 330. In some embodiments, grooves 335 of tip 330 may engage protrusions 326 antenna body 320, and grooves 325 of antenna body 320 may engage with protrusions 336 of tip 330 in an interlocking arrangement. FEP or another sealant can be melted around the grooved portion of the tip 330, melting into the grooves 335 to secure the tip 330 to the antenna body 320. In some embodiments, grooves 335 are interior grooves. In some embodiments, grooves 335 are exterior grooves. By overlapping the FEP over the connecting surfaces of the proximal surface of the tip 330 and the distal surface of the antenna body 320, the FEP can maintain a fluid seal. In some embodiments, the tip 330 comprises grooves 335 and the antenna body 320 comprises grooves 325. In some embodiments, the tip 330 and the antenna body 320 are joined with a sealant to create a fluid tight seal. Various devices and methods for attaching tips to antenna structures are described in PCT patent application PCT/US2019/024564 filed Mar. 28, 2019, disclosing “Systems and Methods Related to Flexible Antennas”, which is incorporated herein by reference in its entirety.

As noted above, tip 330 may be any of the tips described above (e.g., device tips 100, 1100, 2100, 3100, 4100, 5100, 200, 1200, 2200, 3200, 4200, 5200). In some embodiments, tip 330 is constructed of metal or plastic (e.g., PEEK). Where the tip 330 is metal, tip 330 may be electrically attached to the conductive tube of antenna body 320. In some embodiments, the tip 330 is electrically isolated from the conductive tube. The tip 330 could be cylindrically shaped or faceted. Changing the tip 330 from a conductive material to a non-conductive material can change the forward throw of the electromagnetic field formed by the ablation instrument 300, e.g., how far beyond the tip in a distal direction energy is delivered.

The described examples are illustrative, but not limiting, of the present disclosure. Other suitable modifications and adaptations of the variety of conditions and parameters normally encountered in the field, and which would be apparent to those skilled in the art, are within the spirit and scope of the disclosure.

The foregoing description of the specific embodiments will so fully reveal the general nature of the disclosure that others can, by applying knowledge within the skill of the art, readily modify and/or adapt for various applications such specific embodiments, without undue experimentation, and without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance herein.

The breadth and scope of the present disclosure should not be limited by any of the above-described exemplary embodiments, but should be defined only in accordance with the claims and their equivalents.

1-20. (canceled)

21. An ablation instrument comprising:
a cable;
a conductive antenna body coupled to the cable and configured to deliver ablative energy to tissue; and
a tip having a cross-sectional diameter of less than 5 mm,
wherein the tip comprises a blade configured to cut a slit in the tissue, wherein the blade comprises a plurality of cutting edges, and wherein each cutting edge of the plurality of cutting edges has a width between 30% and 50% of the cross-sectional diameter of the tip.

22. The ablation instrument of claim 21, wherein the tip comprises a cone shaped body, and wherein the blade is partially disposed in the cone shaped body.

23. The ablation instrument of claim 21, wherein the tip comprises grooves and protrusions, and wherein the conductive antenna body comprises grooves and protrusions.

24. The ablation instrument of claim 23, wherein the protrusions of the conductive antenna body are configured to engage the grooves of the tip, and wherein the protrusions of the tip are configured to engage the grooves of the conductive antenna body.

25. The ablation instrument of claim 23, wherein the grooves of the tip are axially spaced apart from each other along a longitudinal axis of the tip, and wherein respective pairs of grooves of the tip are separated by respective protrusions of the tip.

26. The ablation instrument of claim 23, wherein the grooves of the conductive antenna body are axially spaced apart from each other along a longitudinal axis of the conductive antenna body, and wherein respective pairs of grooves of the conductive antenna body are separated by respective protrusions of the conductive antenna body.

27. The ablation instrument of claim 21, wherein the tip and the conductive antenna body are configured to be joined with a sealant to create a fluid tight seal.

28. The ablation instrument of claim 21, further comprising:
a jacket including a lumen configured to receive the cable, the conductive antenna body, and the tip.

29. The ablation instrument of claim 28, wherein the jacket is configured to couple the conductive antenna body to the tip.

30. The ablation instrument of claim 28, wherein the conductive antenna body comprises grooves, and wherein the jacket is configured to engage the grooves of the conductive antenna body.

31. The ablation instrument of claim 30, wherein the grooves of the conductive antenna body are axially spaced apart from each other along a longitudinal axis of the conductive antenna body.

32. The ablation instrument of claim 28, wherein the conductive antenna body and the jacket are configured to be joined with a sealant to create a fluid tight seal.

33. The ablation instrument of claim 28, wherein the tip comprises grooves, and wherein the jacket is configured to engage the grooves of the tip.

34. The ablation instrument of claim 33, wherein the grooves of the tip are axially spaced apart from each other along a longitudinal axis of the tip.

35. The ablation instrument of claim 21, wherein the tip is a high temperature plastic, and wherein the blade is a metal.

36. The ablation instrument of claim 21, wherein the tip is a metal and is electrically coupled to the conductive antenna body.

37. The ablation instrument of claim 21, wherein the tip is electrically isolated from the conductive antenna body.

38. The ablation instrument of claim 21, wherein a width of the blade is equal to the cross-sectional diameter of the tip.

39. The ablation instrument of claim 21, wherein each cutting edge of the plurality of cutting edges of the blade is formed by adjacent faces of a plurality of faces of the blade, wherein a first plane intersects each cutting edge of the plurality of cutting edges, and wherein the first plane intersects a longitudinal axis of the tip.

40. The ablation instrument of claim 39, wherein the blade further comprises a plurality of lateral edges, wherein a second plane intersects each lateral edge of the plurality of lateral edges, and wherein the second plane intersects the longitudinal axis of the tip and is radially offset from the first plane.
